(19) United States
(12) Patent Application Publication
(10) Pub. No.: US 2005/0090720 A1
(43) Pub. Date: Apr. 28, 2005

(54) Pulse analyzing apparatus

(76) Inventors: Hsien-Tsai Wu, Tainan (TW); Kai-Chih Chi, Tainan (TW); Kurson Chen, Kao Hsiung (TW)

Correspondence Address: CHARLES E. BAXLEY, ESQ., 90 JOHN STREET, THIRD FLOOR, NEW YORK, NY, 10038, US

(21) Appl. No.: 10/854,093

(22) Filed: May 25, 2004

(30) Foreign Application Priority Data
Oct. 22, 2003 (TW) 092129297

(51) Int. Cl. A61B 5/02

(52) U.S. Cl. 600/300; 600/324

(57) ABSTRACT

A pulse analyzing apparatus includes a measuring unit, a capture unit for processing a pulse signal from the measuring unit, and an operation analyzing unit for calculating the pulse signal processed by the capture unit. Thus, the pulse analyzing apparatus uses a multi-way measurement process to simultaneously measure the pulse signals of different portions of a tested person, thereby simplifying the measurement process and saving the time.

### BACKGROUND OF THE INVENTION

1. Field of the Invention

The present invention relates to a pulse analyzing apparatus, and more particularly to a pulse analyzing apparatus that is measured exactly in an optical manner.

2. Description of the Related Art

The pulse wave velocity (PWV) is the primary standard basis for testing the syndrome of arteriosclerosis. The PWV is used to judge the level of angiosclerosis of the artery by measuring the speed of the blood pulse transmitted to the hand and the foot of a tested person. The PWV of the tested person is defined as the ratio of the conducting distance (Δl) of the pulse and the conducting time (Δt) of the pulse, that V)
PWV=Δl/ΔΔ [equation 1]

A conventional pulse measurement apparatus 9 made by the Tonometry manufacturer in accordance with the prior art shown in FIGS. 7 and 8 uses a oneway measurement process and comprises a Doppler probe 91 which is used to measure the pulse signal 81 of the carotid artery of a tested person and then to measure the pulse signal 82 of the femoral artery of the tested person. Then, the time differential (Δt) between the pulse signal 81 o V the carotid artery and the pulse signal 82 of the femoral artery is located and obtained by a signal 83 measured by an electrocardiogram (ECG) so as to calculate the pulse wave velocity (PWV) of the tested person.

However, the conventional pulse measurement apparatus 9 has the following disadvantages.

1. The conventional pulse measurement apparatus 9 needs aid of a trained and experienced professional person to measure the pulse signals so as to obtain a steady waveform, so that the conventional pulse measurement apparatus 9 is not available for an ordinary user.

2. The conventional pulse measurement apparatus 9 measures the pulse signals by contact, so that measurement of the pulse signals is not objective, thereby decreasing exactness of the measurement.

3. The conventional pulse measurement apparatus 9 needs aid of the ECG, thereby consuming time and increasing costs.

4. The tested person needs to take off the pants for measurement of the femoral artery and needs to being coated with conductive paste for operation of the ECG, thereby causing inconvenience to the tested person.

### SUMMARY OF THE INVENTION

The primary objective of the present invention is to provide a pulse analyzing apparatus that uses a multi-way measurement process to measure the pulse signals of different portions of a tested person simultaneously, thereby simplifying the measurement process and saving the time.

Another objective of the present invention is to provide a pulse analyzing apparatus that is measured exactly in an optical manner.

A further objective of the present invention is to provide a pulse analyzing apparatus that is simple and objective, thereby greatly reducing the time required for measuring the PWV value of the tested person.

A further objective of the present invention is to provide a pulse analyzing apparatus that is operated easily and conveniently without needing aid of a professional person, thereby facilitating a user operating the pulse analyzing apparatus.

A further objective of the present invention is to provide a pulse analyzing apparatus that is operated without needing aid of the ECG and an external instrument, thereby saving time and costs.

In accordance with one embodiment of the present invention, there is provided a pulse analyzing apparatus, comprising:
a measuring unit including a first measuring member mounted on a first portion of a tested person to measure a first pulse signal information of the first portion of the tested person and a second measuring member mounted on a second portion of the tested person to measure a second pulse signal information of the second portion of the tested person, a time differential being defined between the first pulse signal information and the second pulse signal information, and a conducting distance being defined between the first portion and the second portion of the tested person;
a capture unit connected to the measuring unit to capture the first pulse signal information measured by the first measuring member of the measuring unit and the second pulse signal information measured by the second measuring member of the measuring unit simultaneously; and
an operation analyzing unit connected to the capture unit to standardize the first pulse signal information and the second pulse signal information and to perform an operation on the time differential and the conducting distance to calculate a pulse wave velocity of the tested person.

In accordance with another embodiment of the present invention, there is provided a pulse analyzing apparatus for analyzing a first pulse signal information and a second pulse signal information obtained from a first portion and a second portion of a tested person respectively, a time differential being defined between the first pulse signal information and the second pulse signal information, and a conducting distance being defined between the first portion and the second portion of the tested person, the pulse analyzing apparatus comprising:
a program software including means for providing a filtering, gain and digital processing work to the first pulse signal information and the second pulse signal information to produce a processed information, means for locating wave crests and wave troughs of the processed information according to a predetermined threshold and calculating starting points of the first portion and second portion of the tested person, and means for performing an operation on the time differential and the conducting distance to calculate a pulse wave velocity of the tested person.

In accordance with another embodiment of the present invention, there is provided a pulse analyzing method for analyzing a first pulse signal information and a second pulse signal information obtained from a first portion and a second portion of a tested person respectively, a time differential being defined between the first pulse signal information and the second pulse signal information, and a conducting distance being defined between the first portion and the second portion of the tested person, the pulse analyzing method comprising:
providing a filtering, gain and digital processing work to the first pulse signal information and the second pulse signal information to produce a processed information;
locating wave crests and wave troughs of the processed information according to a predetermined threshold and calculating starting points of the first portion and second portion of the tested person; and
performing an operation on the time differential and the conducting distance to calculate a pulse wave velocity of the tested person.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a pulse analyzing apparatus in accordance with the preferred embodiment of the present invention;

FIG. 2 is a side plan cross-sectional view of a first measuring member of the pulse analyzing apparatus as shown in FIG. 1;

FIG. 3 is a side plan view of a clip member of the first measuring member of the pulse analyzing apparatus as shown in FIG. 2;

FIG. 4 is a block view of the pulse analyzing apparatus in accordance with the preferred embodiment of the present invention;

FIG. 5 is a waveform view showing the time differential (Δt) between the first pulse signal information and the second pulse signal information of the pulse analyzing apparatus in accordance with the preferred embodiment of the present invention;

FIG. 6 is a graph showing related curves between the PWV values (DVP-PWV) of the present invention and the PWV values (STD-PWV) of the conventional Tonometry instrument;

FIG. 7 is a perspective view of a conventional pulse measurement apparatus in accordance with the prior art; and

FIG. 8 is a waveform view showing the PWV calculation manner of the conventional pulse measurement apparatus as shown in FIG. 7.

### DETAILED DESCRIPTION OF THE INVENTION

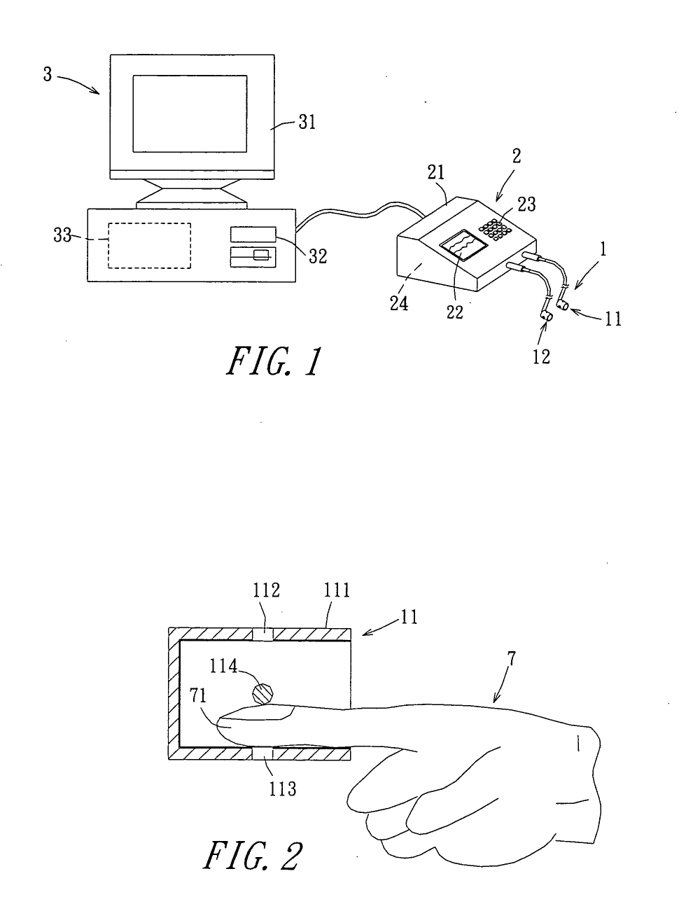

Referring to the drawings and initially to FIGS. 1 and 4, a pulse analyzing apparatus in accordance with the preferred embodiment of the present invention comprises a measuring unit 1, a capture unit 2 connected to the measuring unit 1 for processing a pulse signal from the measuring unit 1, and an operation analyzing unit 3 connected to the capture unit 2 for calculating and converting the pulse signal processed by the capture unit 2.

In the preferred embodiment of the present invention, the measuring unit 1 includes a first measuring member 11 and a second measuring member 12 each connected to the capture unit 2 in a wire connection manner.

The capture unit 2 includes a box 21, an indicator 22 mounted on the box 21, an input interface 23 mounted on the box 21, a first processing module 24 mounted in the box 21 and connected to the first measuring member 11 and the second measuring member 12 of the measuring unit 1, and a memory 25 mounted in the box 21 and connected to the first processing module 24 and the indicator 22. The first processing module 24 of the capture unit 2 includes a filter 241 connected to the measuring unit 1, an amplifier 242 connected to the filter 241, and a digital processor 243 connected to the amplifier 242 and the memory 25.

The operation analyzing unit 3 includes a display 31, a storage device 32 connected to the memory 25 of the capture unit 2 and the display 31, and a second processing module 33 connected to the storage device 32.

It is appreciated that each of the first measuring member 11 and the second measuring member 12 of the measuring unit 1 has the same structure. Thus, only the structure of the first measuring member 11 of the measuring unit 1 is described as follows.

As shown in FIG. 2, the first measuring member 11 of the measuring unit 1 includes a hollow main body 11, an emitter 112 mounted on a first side of the main body 11 for emitting an optical signal, a receiver 113 mounted on a second side of the main body 11 and aligning with the emitter 112 for receiving the optical signal emitted from the emitter 112, and a press portion 114 mounted in the main body 11 for positioning a portion to be measured. Preferably, the press portion 114 is a threaded rod fixed in the main body 11. In the preferred embodiment of the present invention, the optical signal is transmitted by infrared rays.

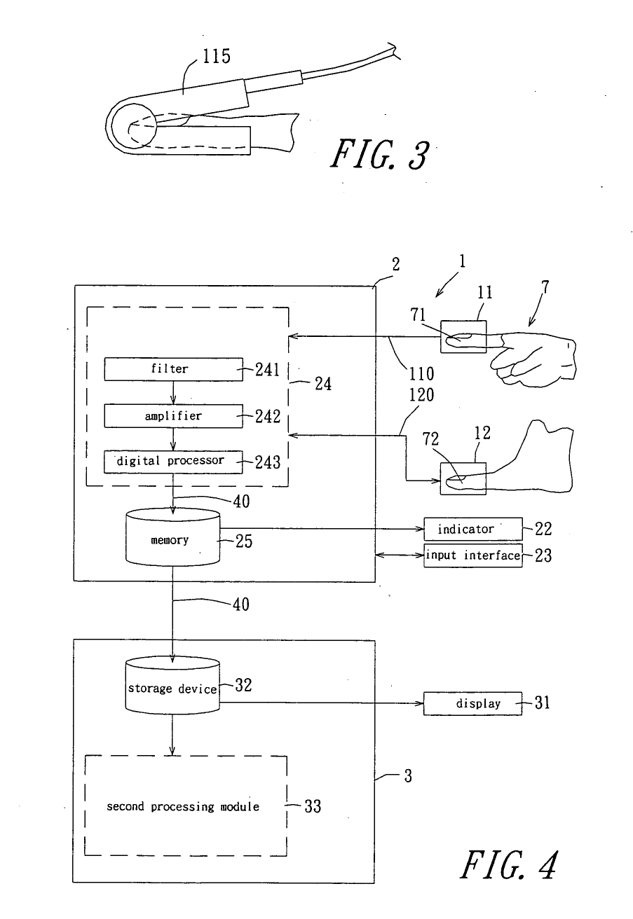

As shown in FIG. 3, the press portion 114 is replaced by a clip member 115 for positioning a portion to be measured, so that the measuring unit 1 is available measured portions having different sizes.

Again referring to FIG. 2, when a first portion 71 (such as one finger) of a tested person 7 is extended into the inside of the main body 11, the first portion 71 of the tested person 7 is pressed by the press portion 114, and the emitter 112 emits an infrared optical signal which passes through the first portion 71 of the tested person 7 and is received by the receiver 113. At this time, when the infrared optical signal which passes through the first portion 71 of the tested person 7, the blood flow rate contained in the first portion 71 of the tested person 7 is changed due to variation of the heart beat, thereby changing the optical permeability in the blood, so that the infrared optical signal received by the receiver 113 is also changed accordingly. Thus, the measuring unit 1 can measure the pulse signal of the first portion 71 (one finger) of the tested person 7.

Referring to FIGS. 1 and 4, the pulse analyzing apparatus is used to measure the values of the pulse wave velocity (PWV). After the tested person 7 is situated at a stationary state during a period of time about five to ten minutes, the first measuring member 11 and the second measuring member 12 of the measuring unit 1 are respectively mounted on the first portion 71 (one finger of the right hand) and the second portion 72 (one toe of the right foot) of the tested person 7 at the same side so as to measure a first pulse signal information 110 of the first portion 71 of the tested person 7 and a second pulse signal information 112 of the second portion 72 of the tested person 7 simultaneously.

Then, the first pulse signal information 110 and the second pulse signal information 112 of the tested person 7 are transmitted by the measuring unit 1 to the capture unit 2. Then, the first pulse signal information 110 and the second pulse signal information 112 of the tested person 7 are transmitted through the filter 241 of the capture unit 2 for filtering the pulse noise, then through the amplifier 242 of the capture unit 2 for obtaining a gain of the pulse signals and then through the digital processor 243 which performs a sampling process according to the sample frequency of 200 Hz, thereby obtaining a digital volume pulse (DVP) signal 40. Then, the DVP signal 40 of the tested person 7 is stored in the memory 25 of the capture unit 2 and indicated by the indicator 22 of the capture unit 2. Then, the DVP signal 40 of the tested person 7 is transmitted to the operation analyzing unit 3 in the RS232 serial transmission manner to analyze the DVP signal 40 of the tested person 7 by the operation analyzing unit 3.

In practice, the filter 241 of the capture unit 2 is used to filter the noise frequency of 60 Hz produced by the normal electric power. Usually, the pulse signals contain direct current signals and alternating current signals whose amplitudes are smaller than that of the direct current signals. Thus, the filter 241 of the capture unit 2 is used to filter the direct current signals to leave the alternating current signals to react variation of the pulse signals. In addition, the capture unit 2 employs a micro processor chip module to function as its control center. In the preferred embodiment of the present invention, the micro processor chip module is the MSP430 mixing signal micro processor produced by the TI (Texas instrument) company. The functions of the filter 241, the amplifier 242 and the digital processor 243 of the capture unit 2 are conventional and will not be further described in detail.

After the operation analyzing unit 3 receives the DVP signal 40 of the tested person 7 from the capture unit 2, the storage device 32 and the second processing module 33 of the operation analyzing unit 3 performs a locating work to locate the wave crest, wave trough and starting point of the DVP signal 40 of the tested person 7. In the preferred embodiment of the present invention, the storage device 32 of the operation analyzing unit 3 is a solid memory, optical storage medium (such as laser disc), magnetic storage medium (such as floppy disc or magnetic tape) or the like. in such a manner, the DVP signal 40 received by the operation analyzing unit 3 is stored in the storage device 32 in an array manner. In addition, the second processing module 33 of the operation analyzing unit 3 judges and calculates the main wave crest, heart rates and starting point of the DVP signal 40 at each wave section (during about five seconds).

In practice, the threshold values are used as the judgement basis of the main wave crest and the wave trough.

Assuming the DVP signal 40 is an array x[n] having a length of 1000, the main wave crest and the wave trough are taken from the threshold value. The threshold value is set as the difference between the maximum and the minimum of a waveform of 0.25 times. Thus, the threshold value is set as follows.
Threshold=[Max(x[n])−Min(x[n])]*0.25 [equation 2]

Then, each point is compared with the threshold value as follows.
(Max(x[n])−x[n₁]) Threshold 1≦n₁≦n [equation 3]

The values satisfying the comparison equation 3 are stored in the array y[n]. The maximum points in the array y[n] correspond to different n values which are the main wave crests of the desired x[n].

Similarly, each point is compared with the threshold value as follows.
(x[n₁]−Min(x[n])) Threshold 1≦n₁≦n [equation 4]

The values satisfying the comparison equation 4 are stored in the array z[n] which is the first order derivative array of the array x[n]. The maximum points in the array z[n] correspond to different n values which are the main wave troughs of the desired x[n].

After the main wave crests of all of the periods in the wave are obtained, the interval between any two adjacent main wave crests are used to calculate the hear rate.

Assuming the x-axis values corresponding to all of the main wave crests are stored in an array Maxindex (index), and the index represents the number of all of the main wave crests in the wave, the heart rate is calculated as follows.

$$H . R . = \frac{index * 1 * 60}{{\sum }^{index - 1} ( Max index ( i + 1 ) - Max index ( i ) ) * 0.005}$$

The number 0.005 is the inverse (1/200 Hz) of the sample frequency 200 Hz, which indicates that the distance between any two adjacent sample points is equal to 0.005 s. The equation 5 converts the average heart beat period (the distance between the main wave crests) into a frequency which multiplies 60 to obtain the heart rate which means the heart beat number every minute.

The main wave crest and the wave trough of each set are used as the judgement basis of the starting point. The starting point has two primary features including: the slope has the maximum variation and the rising altitude after the starting point reaches the maximum value.

The second processing module 33 of the operation analyzing unit 3 initially calculates the slope variation of every five points between the wave trough and the main wave crest (the slope variation of only one point is easily misjudged due to noise).

Thus, the slope variation of every five points is stored in an array of Pacemaker, and the second comparison condition exists in the array of compare (i) as follows.
compare(i)=x[Pacemaker(i)+30]−x[Pacemaker(i)] 1≦i≦5 [equation 6]

In such a manner, the maximum value in the array of compare (i) is the desired starting point. In addition, by means of analyzing the starting point in the waveform, the conducting time is obtained by comparing the time differential (Δt) between the starting points of the finger and the toe.

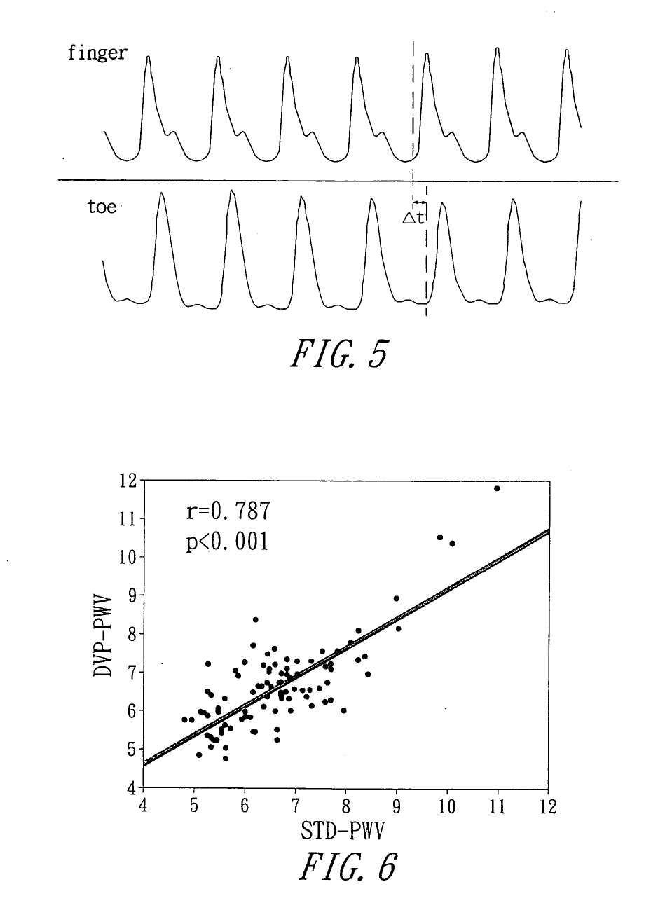

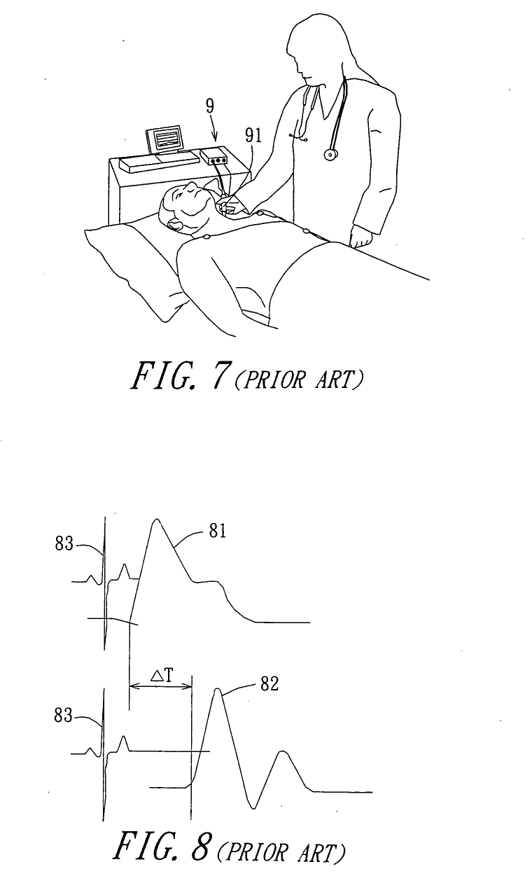

As shown in FIGS. 4 and 5, the first pulse signal information 110 and the second pulse signal information 112 of the tested person 7 are produced simultaneously, so that the DVP signals 40 output by the first pulse signal information 110 and the second pulse signal information 112 are calculated by the operation analyzing unit 3 to obtain the time differential (Δt) between the first pulse signal information 110 and the second pulse signal information 112. In the preferred embodiment of the present invention, the conducting distance (ΔN) is defined as the difference between the vertical distance of the first portion 71 (one finger of the right hand) of the tested person 7 to the carotid artery and the vertical distance of the second portion 72 (one toe of the right foot) of the tested person 7 to the carotid artery. Then, the conducting distance (ΔN) is input into the capture unit 2 through the input interface 25. Finally, the operation analyzing unit 3 performs an operation on the time differential (Δt) and the conducting distance (Δl) so as to obtain the pulse N1) so as to obtain in the blood of the tested person 7.

In experiment, the PWV measurement method (DVP-PWV) of the present invention is compared with the PWV measurement method (STD-PWV) of the conventional Tonometry instrument as follows.

In the first experiment, the conventional Tonometry instrument uses a oneway measurement method which uses a Doppler probe to measure the pulse signal of the carotid artery and the pulse signal of the femoral artery. Then, the time differential between the pulse signals of the carotid artery and the femoral artery is measured by an electrocardiogram (ECG) so as to calculate the PWV value (STD-PWV).

In the second experiment, the pulse analyzing apparatus of the present invention is used to calculate the PWV value (DVP-PWV).

As shown in FIG. 6, the experimental results show that the PWV measurement method (DVP-PWV) of the present invention is highly related to the PWV measurement method (STD-PWV) of the conventional Tonometry instrument, that is, relation R is equal to 0.787.

In addition, the PWV measurement method (DVP-PWV) of the present invention is compared with the PWV measurement method (STD-PWV) of the conventional Tonometry instrument in the table 1 as follows.

| | DVP-PWV | STD-PWV |
|---|---|---|
| Age | R = 0.401 | R = 0.458 |
| | P < 0.001 | P < 0.001 |
| SBP | R = 0.455 | R = 0.501 |
| | P < 0.001 | P < 0.001 |
| DBP | R = 0.463 | R = 0.541 |
| | P < 0.001 | P < 0.001 |

Note:
SBP: Systolic Blood Pressure
DBP: Diastolic Blood Pressure
P < 0.001 indicates the difference exists without relation to the probability.

As shown in the table 1, the age of the tested person 7 is highly related to the PWV measurement method (DVP-PWV) of the present invention, that is, the relation R is equal to 0.401, which indicates that the blood vessel is aged with increase of the age of the tested person 7, and the PWV value is increased accordingly. Thus, the relation R of the DVP-PWV is highly related to that of the STD-PWV in the age, the SBP and the DBP.

In addition, the PWV value is measured in the test table 2 as follows.

| | DVP-PWV | STD-PWV |
|---|---|---|
| Hypertension + (10) | 8.04 ± 1.83 | 8.14 ± 1.47 |
| Hypertension − (90) | 6.49 ± 0.92 | 6.51 ± 1.01 |
| P | <0.001 | 0.007 |

As shown in the table 2, hypertension is the danger factor of arteriosclerosis, so that the PWV value of the tested person 7 subjected to the hypertension is much greater than that of the normal person.

In addition, the P value of the DVP-PWV is smaller than that of the STD-PWV, which indicates that the pulse analyzing apparatus of the present invention has greater exactness.

In conclusion, the pulse analyzing apparatus of the present invention has the following advantages.

1. The pulse analyzing apparatus is simple and objective, thereby greatly reducing the time required for measuring the PWV value of the tested person.

2. The pulse analyzing apparatus is operated easily and conveniently without needing aid of a professional person, thereby facilitating a user operating the pulse analyzing apparatus.

3. The pulse analyzing apparatus is operated without needing aid of the ECG and an external instrument, thereby saving time and costs.

4. The pulse analyzing apparatus measures the DVP signals of the finger and the toe of the tested person simultaneously, so that the pulse analyzing apparatus uses a multi-way measurement process to measure the PWV value of the tested person, thereby measuring the time differential of the pulse exactly.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

1. A pulse analyzing apparatus, comprising:
a measuring unit including a first measuring member mounted on a first portion of a tested person to measure a first pulse signal information of the first portion of the tested person and a second measuring member mounted on a second portion of the tested person to measure a second pulse signal information of the second portion of the tested person, a time differential being defined between the first pulse signal information and the second pulse signal information, and a conducting distance being defined between the first portion and the second portion of the tested person;
a capture unit connected to the measuring unit to capture the first pulse signal information measured by the first measuring member of the measuring unit and the second pulse signal information measured by the second measuring member of the measuring unit simultaneously; and
an operation analyzing unit connected to the capture unit to standardize the first pulse signal information and the second pulse signal information and to perform an operation on the time differential and the conducting distance to calculate a pulse wave velocity of the tested person.

2. The pulse analyzing apparatus in accordance with claim 1, wherein the first measuring member of the measuring unit measures the first pulse signal information by emitting and receiving an optical signal passing through the first portion of the tested person, and the second measuring member of the measuring unit measures the second pulse signal information by emitting and receiving an optical signal passing through the second portion of the tested person.

3. The pulse analyzing apparatus in accordance with claim 1, wherein the first portion and the second portion of the tested person are located at the same side of the tested person.

4. The pulse analyzing apparatus in accordance with claim 3, wherein the first portion of the tested person is one finger of the tested person, and the second portion of the tested person is one toe of the tested person at the same side.

5. The pulse analyzing apparatus in accordance with claim 4, wherein the conducting distance is defined as a difference between a vertical distance of the finger of the tested person to the carotid artery and a vertical distance of the toe of the tested person to the carotid artery.

6. The pulse analyzing apparatus in accordance with claim 1, wherein the capture unit includes a first processing module to provide a filtering, gain and digital processing work to the first pulse signal information and the second pulse signal information.

7. The pulse analyzing apparatus in accordance with claim 1, wherein the operation analyzing unit includes a second processing module to locate wave crests, wave troughs and starting points of the first pulse signal information and the second pulse signal information and to calculate a heart rate and the pulse wave velocity of the tested person.

8. A pulse analyzing apparatus for analyzing a first pulse signal information and a second pulse signal information obtained from a first portion and a second portion of a tested person respectively, a time differential being defined between the first pulse signal information and the second pulse signal information, and a conducting distance being defined between the first portion and the second portion of the tested person, the pulse analyzing apparatus comprising:
a program software including means for providing a filtering, gain and digital processing work to the first pulse signal information and the second pulse signal information to produce a processed information, means for locating wave crests and wave troughs of the processed information according to a predetermined threshold and calculating starting points of the first portion and second portion of the tested person, and means for performing an operation on the time differential and the conducting distance to calculate a pulse wave velocity of the tested person.

9. The pulse analyzing apparatus in accordance with claim 8, further comprising a storage device connected to the program software to store the pulse wave velocity of the tested person.

10. The pulse analyzing apparatus in accordance with claim 8, further comprising a display connected to the program software to indicate the pulse wave velocity of the tested person.

11. A pulse analyzing method for analyzing a first pulse signal information and a second pulse signal information obtained from a first portion and a second portion of a tested person respectively, a time differential being defined between the first pulse signal information and the second pulse signal information, and a conducting distance being defined between the first portion and the second portion of the tested person, the pulse analyzing method comprising:
providing a filtering, gain and digital processing work to the first pulse signal information and the second pulse signal information to produce a processed information;
locating wave crests and wave troughs of the processed information according to a predetermined threshold and calculating starting points of the first portion and second portion of the tested person; and
performing an operation on the time differential and the conducting distance to calculate a pulse wave velocity of the tested person.
